(19) United States
(12) Patent Application Publication
(10) Pub. No.: US 2026/0193663 A1
(43) Pub. Date: Jul. 9, 2026

(54) SORGHUM DOUBLED HAPLOID BREEDING METHOD

(71) Applicant: INSTITUTE OF CROP SCIENCES, CHINESE ACADEMY OF AGRICULTURAL SCIENCES, Beijing City (CN)

(72) Inventors: Yi SUI, Beijing City (CN); Chuanyin WU, Beijing City (CN); Yao SUN, Beijing City (CN); Zixiang CHENG, Beijing City (CN)

(73) Assignee: INSTITUTE OF CROP SCIENCES, CHINESE ACADEMY OF AGRICULTURAL SCIENCES, Beijing City (CN)

(21) Appl. No.: 19/540,582

(22) Filed: Feb. 14, 2026

Related U.S. Application Data
(63) Continuation of application No. PCT/CN2025/137044, filed on Nov. 24, 2025.

(30) Foreign Application Priority Data
Jan. 7, 2025 (CN) 202510021535.5

(51) Int. Cl. C12N 15/82; C12N 9/22; C12N 15/11

(52) U.S. Cl. CPC C12N 15/8205; C12N 9/226; C12N 15/111; C12N 15/8241; C12N 2310/20

(57) ABSTRACT

A Sorghum doubled haploid breeding method is provided. The method includes following steps: (1) mutating SbMTL and SbDMP genes to obtain the Sorghum double-gene mutant line; (2) using green fluorescent protein (GFP) marker and Ruby marker genes to screen and identify Sorghum haploids; and (3) naturally doubling the obtained haploid plants to obtain Sorghum doubled haploids.

### CROSS-REFERENCE TO RELATED APPLICATIONS

This application is a continuation of International Patent Application No. PCT/CN2025/137044, filed on Nov. 24, 2025, and claims priority of Chinese Patent Application No. 202510021535.5, filed on Jan. 7, 2025. The contents of International Patent Application No. PCT/CN2025/137044 and Chinese Patent Application No. 202510021535.5 are hereby incorporated by reference.

### SEQUENCE LISTING

The present application includes a sequence listing in electronic form as an XML file in XML format, created on Jan. 8, 2026, titled “sequence.xml” and having a size of 18,895 bytes. The contents of this XML file are incorporated by reference herein.

### TECHNICAL FIELD

The present disclosure belongs to the field of biotechnology and agricultural biological breeding technology, and specifically relates to a Sorghum doubled haploid breeding method.

### BACKGROUND

Sorghum is the fifth largest crop globally. Due to its drought and high temperature tolerance, Sorghum is suitable for cultivation in regions where maize, rice, and wheat struggle to grow. Sorghum is not only an important food source in these areas but also widely used in various fields such as brewing (alcohol, vinegar), ethanol production, and silage, making it one of the ideal crops for addressing global climate change.

Conventional Sorghum breeding has long cycles and high costs, while modern breeding technologies such as Doubled Haploid (DH) technology have been successfully applied in maize because they may rapidly fix recombinant haplotypes. However, their application in Sorghum remains lagging. Current research has identified two natural haploid induction lines, SMHI01 and SMHI021, from 4,000 Sorghum germplasms, with a haploid induction efficiency of only 1-2% (Hussain and Franks 2019). In the patent CN110546266A by KWS SAAT SE & Co. KGaA, haploidization in Sorghum is disclosed, achieving only a 1% induction efficiency. Such low induction efficiency limits the application of doubled haploid breeding technology in Sorghum, failing to meet the required induction rate of over 10% for large-scale breeding. Therefore, improving the haploid induction frequency in Sorghum is crucial.

Maize haploid induction originated from the Stock6 germplasm, and the two major cloned quantitative trait loci (QTLs) are the phospholipase gene MATRILINEAL/Zea mays Phospholipase A1/Not Like Dad (MATL/ZmPLA1/NLD) and the Zea mays DUF679 domain membrane protein (ZmDMP) gene. Mutating the phospholipase gene alone may achieve an induction efficiency of approximately 2-3%, while mutating the ZmDMP gene on this basis may increase the induction rate to 8-15% (Zhong, Liu et al. 2019), indicating that MATRILINEAL (MTL) and ZmDMP have a synergistic effect on haploid induction in maize. It has been confirmed that in monocots, single mutation of the MTL gene may achieve haploid induction in rice, foxtail millet, and wheat, with an efficiency of about 3% (Yao, Zhang et al. 2018, Liu, Zhong et al. 2020, Cheng, Sun et al. 2021). In dicots, due to the lack of conservation of the MTL gene, knocking out ZmDMP homologous genes in various dicots only results in an induction rate of about 3%, while in maize, the induction rate related to zmdmp is only 0.1-0.3%. Conversely, further exploration in rice by knocking out OsDMP3 and OsDMP6 revealed that they themselves have neither haploid induction ability nor a synergistic effect with osmtl on the induction rate. Currently, apart from maize, no other crop has achieved a maternal haploid induction frequency of 8% or higher required for acceptable commercial-scale doubled haploid breeding. The induction efficiency by mutating the Sorghum SbMTL gene via the Tilling method in a German patent is only 1%, far from meeting the requirements for industrialized DH breeding.

Therefore, whether mutating the Sorghum SbMTL and SbDMP genes individually or simultaneously may achieve high-frequency induction lines is the primary problem to be solved by the present disclosure. Simultaneously, the present disclosure is also dedicated to establishing effective markers for screening haploids, thereby providing an effective solution for the industrialization of Sorghum doubled haploid breeding.

### SUMMARY

The objective of the present disclosure is to solve the aforementioned problems existing in the prior art. A Sorghum doubled haploid breeding method is proposed, which utilizes clustered regularly interspaced short palindromic repeats/CRISPR-associated protein 9 (CRISPR/Cas9) technology to knock out Sorghum SbMTL and SbDMP genes to achieve high-frequency haploid induction, while introducing effective haploid screening markers, and evaluates the induction frequency of haploid induction lines. After natural doubling of the obtained haploid plants, Sorghum doubled haploid lines are successfully obtained, significantly advancing the process of Sorghum breeding.

The technical scheme of the present disclosure is as follows.

The present disclosure provides a Sorghum double-gene mutant line. The Sorghum double-gene mutant line is obtained by knocking out Sorghum SbMTL and SbDMP genes using CRISPR/Cas9 technology in P89GFP and TxGFP backgrounds, respectively. The obtained SbMTL and SbDMP double-gene mutant lines include two knockout lines: sbdmp-1 sbmtl-1; TxGFP and sbdmp-2 sbmtl-2; P89GFP.

In an embodiment, the construction method for the Sorghum double-gene mutant line includes the following steps:
selecting a target site SbMTL-sgRNA1 with the sequence as shown in SEQ ID NO.13 and a target site SbMTL-sgRNA2 with the sequence as shown in SEQ ID NO.14 on a Sorghum SbMTL genome; and selecting a target site SbDMP-sgRNA1 with the sequence as shown in SEQ ID NO.15 on a Sorghum SbDMP genome;
constructing CRISPR/SbMTL-Cas9-1, CRISPR/SbMTL-Cas9-2, and CRISPR/SbDMP-Cas9-1 vectors including target site DNA sequences; and
using Agrobacterium to transform the vectors into Sorghum P89GFP and TxGFP receptors to obtain transgenic Sorghum with knocked-out SbMTL and SbDMP genes, thereby obtaining the Sorghum double-gene mutant line.

In an embodiment, in the P89GFP background, the SbDMP gene on SbMTL single mutant plants is knocked out to obtain sbmtl sbdmp double-gene mutant haploid induction lines.

The present disclosure also provides the application of the Sorghum double-gene mutant line according to any one of the above items in Sorghum doubled haploid breeding.

The present disclosure also provides a Sorghum doubled haploid breeding method, including the following steps:
(1) mutating SbMTL and SbDMP genes to obtain the Sorghum double-gene mutant line according to any one of claims 1 to 3;
(2) using green fluorescent protein (GFP) marker and Ruby marker genes to screen and identify Sorghum haploids; and
(3) naturally doubling obtained haploid plants to obtain Sorghum doubled haploids.

In an embodiment, fluorescent marker lines P89GFP and TxGFP containing the GFP marker are created. In line P89GFP, the transfer DNA (T-DNA) insertion site is located at position 75,331,531 on chromosome 2. In line TxGFP, the T-DNA insertion site is located at position 4,617,987 on chromosome 1.

The beneficial effects of the present disclosure are as follows.

The present disclosure achieves high-frequency induction lines by simultaneously mutating SbMTL and SbDMP genes, and establishes effective markers for screening haploids, that is, creating Sorghum single-copy marker gene germplasm through the GFP reporter gene and Ruby marker gene, evaluating its phenotypic differences from the wild type, then mutating SbMTL and SbDMP genes to create induction line germplasm containing markers. Finally, through natural doubling of haploids produced by the high-frequency induction lines, Sorghum doubled haploids may be obtained in a short period, rapidly applied to new variety breeding, providing an effective solution for the industrialization of Sorghum doubled haploid breeding, and also offering an application case for the development of doubled haploid breeding technology in other species.

Using the Sorghum haploid induction line of the present disclosure for doubled haploid breeding significantly accelerates the breeding process, and the induction line itself is a key technical point in the entire doubled haploid breeding procedure. Currently, due to the limitations of genetic transformation technology bottlenecks both domestically and internationally, Sorghum haploid induction lines have not yet been developed, while the method of the present disclosure solves all key technical difficulties in Sorghum doubled haploid breeding. In particular, the Sorghum haploid induction line of the present disclosure has an induction efficiency as high as above 18%, possessing extremely high economic value.

### BRIEF DESCRIPTION OF THE DRAWINGS

FIG. 1A, FIG. 1B, FIG. 1C, FIG. 1D and FIG. 1E show the development of Sorghum single-copy fluorescent marker lines.

FIG. 1A is a schematic diagram of the GFP-H and GFP-B vectors.

FIG. 1B shows seeds of the T₂ generation TxGFP stably expressing fluorescence signals under white light (left) and ultraviolet light (right).

FIG. 1C shows seeds of the T₂ generation P89GFP stably expressing fluorescence signals under white light (left) and ultraviolet light (right).

FIG. 1D shows that TxGFP carries the GFP-H insertion on chromosome 1 (Chr1:4617987).

FIG. 1E shows that the P89GFP line carries the GFP-B insertion on chromosome 2 (Chr2:75331531).

FIG. 2A, FIG. 2B, FIG. 2C and FIG. 2D show the verification of the usability of single-copy green fluorescent protein (GFP) marker lines.

FIG. 2A shows the determination of an effective fluorescent selection marker by comparing the wild type (GFP-), two-copy GFP (GFP-homo) from TxGFP, and single-copy (GFP-heter) seeds derived from male sterile line (L407A)×TxGFP or P89GFP.

FIG. 2B shows the determination of an effective fluorescent selection marker by comparing the wild type (GFP-), two-copy GFP (GFP-homo) from P89GFP, and single-copy (GFP-heter) seeds derived from male sterile line (L407A)×TxGFP or P89GFP.

FIG. 2C shows a comparison of the phenotypic characteristics of the TxGFP line with its respective wild type.

FIG. 2D shows a comparison of the phenotypic characteristics of the P89GFP line with its respective wild type.

FIG. 3A and FIG. 3B show information on the genomic structure of SbMTL and SbDMP genes, clustered regularly interspaced short palindromic repeats/CRISPR-associated protein 9 (CRISPR/Cas9) targets, and obtained mutant plants.

FIG. 3A shows information on two target sites on the SbMTL gene, respectively obtaining editing types of two homozygous frameshift mutant lines.

FIG. 3B shows information on a single target site on the SbDMP gene, obtaining editing types of single mutant lines and double mutants.

FIG. 4 shows the phenotype of Sorghum hybrid-induced haploid plants.

FIG. 5A and FIG. 5B show a schematic diagram of flow cytometry results.

FIG. 5A shows the detection results of diploid.

FIG. 5B shows the detection results of haploid.

FIG. 6A and FIG. 6B show a schematic diagram of Sorghum haploid identification.

FIG. 6A shows a comparison under white light between Sorghum diploids (D) and haploids (H) after pollination with the induction line.

FIG. 6B shows a comparison of diploids (D) and haploids (H) under ultraviolet light.

FIG. 7A, FIG. 7B and FIG. 7C show a schematic diagram of identifying Sorghum haploids using Ruby markers.

FIG. 7A is a schematic diagram of the expression vector structure of Ruby transformed into the induction line.

FIG. 7B demonstrates the distinction between diploid (D) and haploid (H) by the presence or absence of red markers in the plumule, where the markers in the plumule of the diploid (D) appear as a visually darker region.

FIG. 7C shows that Sorghum seeds after crossing with the induction line are directly sown in soil, and the growing plants may be identified for haploids using the red Ruby marker, with green (a lighter color) indicating haploids (shown by white arrows) and all red plants (a darker color) being diploids.

FIG. 8A and FIG. 8B show seeds of doubled haploids produced by natural doubling of Sorghum haploids.

FIG. 8A shows the panicle of a Sorghum haploid, with individual florets capable of natural doubling to produce seeds of doubled haploids (DH₀).

FIG. 8B is a partial enlargement of FIG. 8A, with seeds of DH₀ indicated by white arrows.

FIG. 9 is a flow chart of the construction method for the Sorghum double-gene mutant line.

FIG. 10 is a flow chart of a Sorghum doubled haploid breeding method.

### DETAILED DESCRIPTION OF THE EMBODIMENTS

The technical schemes in the embodiments of the present disclosure will be clearly and completely described below in conjunction with the accompanying drawings in the embodiments of the present disclosure. It is apparent that the described embodiments are only a part of the embodiments of the present disclosure, and not all of the embodiments. All other embodiments obtained by a person of ordinary skill in the art based on the embodiments of the present disclosure without creative efforts shall fall within the protection scope of the present disclosure.

To further understand the present disclosure, the present disclosure will be further illustrated in combination with the accompanying drawings and embodiments.

The experimental methods in the following embodiments are conventional methods unless otherwise specified, and are carried out according to the techniques or conditions described in the literature in the art or according to the product instructions. The materials, reagents, etc., used in the following embodiments are available from commercial sources unless otherwise specified.

### Embodiment 1

### Acquisition of Sorghum Single-Copy Green Fluorescent Protein (GFP) Lines

An effective haploid screening marker is the basis for later selection in doubled haploid breeding. Based on this, the present disclosure adopts a transgenic method, introducing the GFP gene driven by CaMV35S into Sorghum varieties P898012 and Tx430 via Agrobacterium-mediated genetic transformation, to develop fluorescent marker lines for Sorghum. Specifically, the HYG gene and the BAR gene are used as effective selection markers for Agrobacterium-mediated genetic transformation, respectively. Among 94 independent transgenic events, stable lines with strong GFP expression are selected, and their copy numbers are evaluated by quantitative polymerase chain reaction (PCR). Finally, one line with stable high expression of GFP is screened from each of the P898012 and Tx430 varieties as base materials for subsequent research (hereinafter referred to as P89GFP and TxGFP).

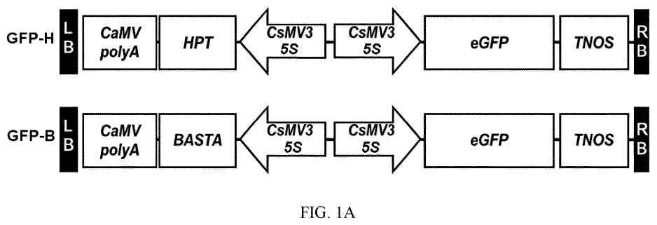

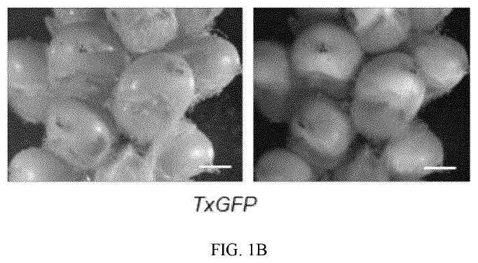

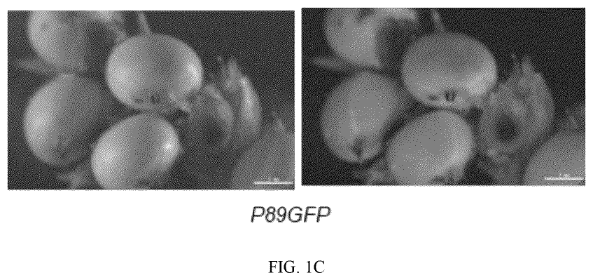

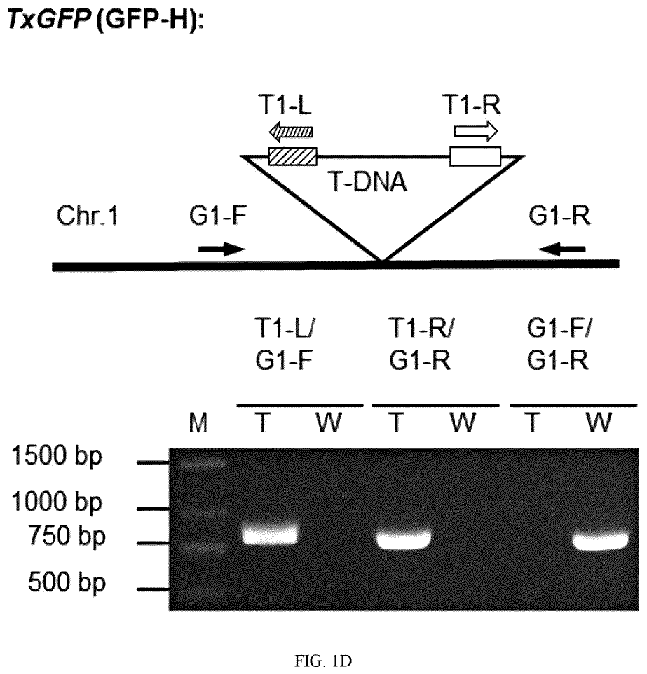

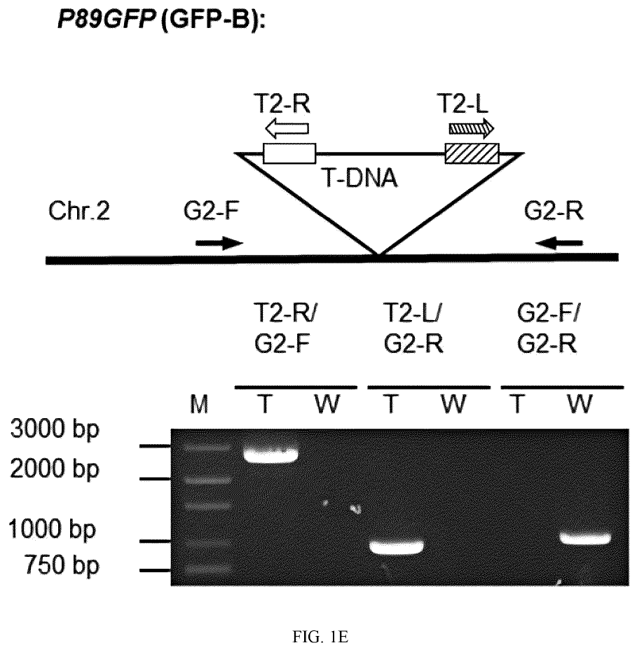

The transfer DNA (T-DNA) insertion sites of the two lines are identified by high-throughput sequencing, and the site information is further confirmed by PCR. In P89GFP, CaMV35S:GFP is located at position 75,331,531 on chromosome 2; and in TxGFP, it is located at position 4,617,987 on chromosome 1, as shown in FIG. 1A, FIG. 1B, FIG. 1C, FIG. 1D and FIG. 1E. FIG. 1A is a schematic diagram of the GFP-H and GFP-B vectors, which are used for Agrobacterium-mediated transformation. FIG. 1B shows seeds of the T₂ generation TxGFP stably expressing fluorescence signals under white light (left) and ultraviolet light (right). FIG. 1C shows seeds of the T₂ generation P89GFP stably expressing fluorescence signals under white light (left) and ultraviolet light (right). FIG. 1D-FIG. 1E confirm the two T-DNA insertion sites, with TxGFP (FIG. 1D) carrying GFP-H inserted into chromosome 1 (Chr1:4617987) and the P89GFP line (FIG. 1E) carrying GFP-B inserted into chromosome 2 (Chr2:75331531). Two primers (black arrows) adjacent to the genomic insertion site and T-R (white arrow) and T-L (black shaded arrow) primers at the T-DNA boundaries are designed for each insertion site. The T-DNA insertion sites for each line are confirmed by electrophoresis gel; where W represents the wild type; T represents the TxGFP or P89GFP line; M represents the molecular weight marker; the white rectangle indicates the T-DNA right border; the black shaded rectangle indicates the left border; and Bar=2 millimeters.

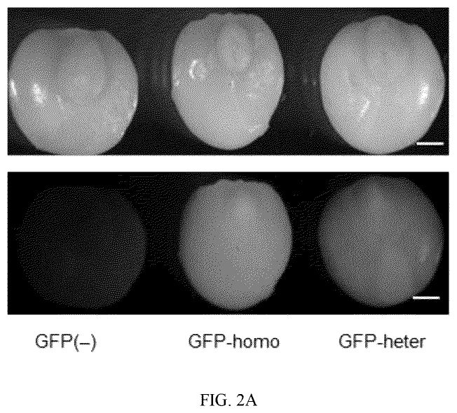

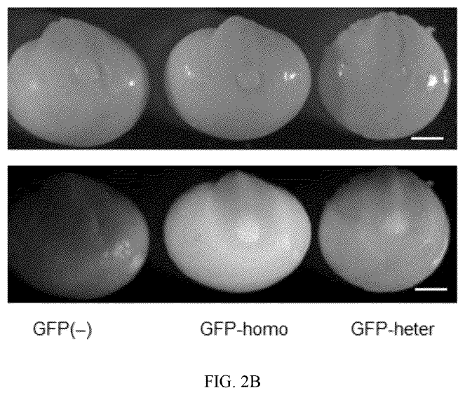

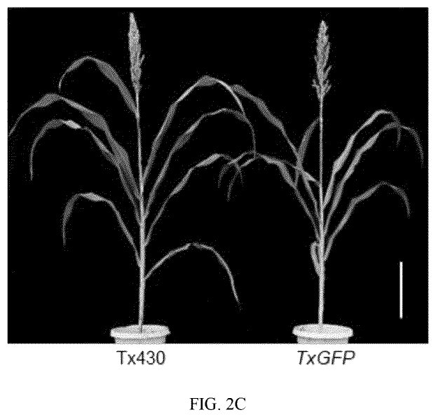

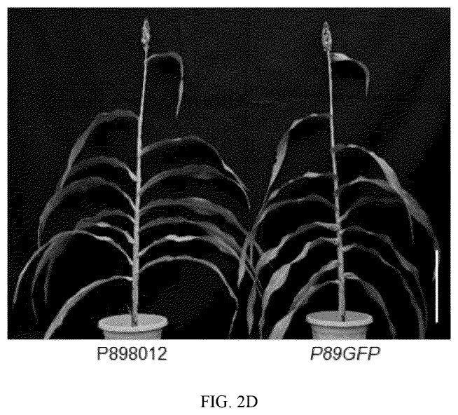

The pollen of these two marker lines is used for pollination with male sterile line L407A to evaluate the practicality of the GFP marker, and consistent GFP signals are observed in all hybrid seeds. FIG. 2A-FIG. 2B show the determination of an effective fluorescent selection marker by comparing the wild type (GFP-), two-copy GFP (GFP-homo) from TxGFP (FIG. 2A) and P89GFP (FIG. 2B), and single-copy (GFP-heter) seeds derived from male sterile line (L407A)×TxGFP or P89GFP. FIG. 2C-FIG. 2D show the comparison of phenotypic characteristics between the TxGFP (FIG. 2C) and P89GFP (FIG. 2D) lines and their respective wild types. As may be seen from FIG. 2A, FIG. 2B, FIG. 2C and FIG. 2D, these two lines show no obvious morphological differences compared to the wild type. Therefore, fluorescent marker lines are successfully created as the first step in Sorghum haploid production.

### Embodiment 2

### Acquisition of Sorghum Haploid Induction Genes SbMTL and SbDMP

Based on the known protein sequences of maize haploid induction genes ZmPLA1/NLD/MATL and ZmDMP, homologous genes in Sorghum are retrieved using the bioinformatics platform (www.phytozome.org) version 12 and are named SbMTL (Sobic.001G348600) and SbDMP (Sobic.010G093200) respectively, where the genomic sequence of SbMTL is as shown in SEQ ID NO.1, and the coding sequence (CDS) is as shown in SEQ ID NO.2; and the genomic sequence of the SbDMP gene is as shown in SEQ ID NO.3, and the CDS sequence is as shown in SEQ ID NO.4. The amino acid sequences of the proteins encoded by the SbMTL and SbDMP genes are as shown in SEQ ID NO.5 and SEQ ID NO.6, respectively.

### Embodiment 3

### Acquisition of Sorghum SbMTL and SbDMP Single-Gene and Double-Gene Mutant Lines Using Clustered Regularly Interspaced Short Palindromic Repeats/CRISPR-Associated Protein 9 (CRISPR/Cas9) Technology

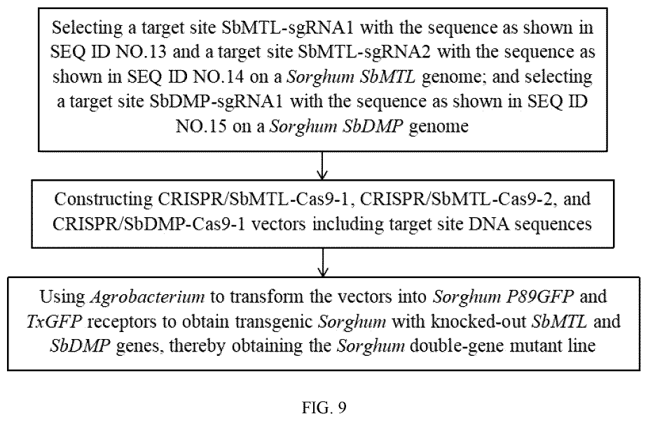

The CRISPR/Cas9 system is used to knock out Sorghum SbMTL and SbDMP genes in the P89GFP and TxGFP backgrounds, respectively, simultaneously, the SbDMP gene is knocked out again on Sbmtl single mutant plants, resulting in six knockout lines: sbmtl-1; TxGFP, sbmtl-2; P89GFP, sbdmp-1; TxGFP, sbdmp-2; P89GFP, sbdmp-1 sbmtl-1; TxGFP, and sbdmp-2 sbmtl-2; P89GFP. As shown in FIG. 9, the specific steps are as follows.

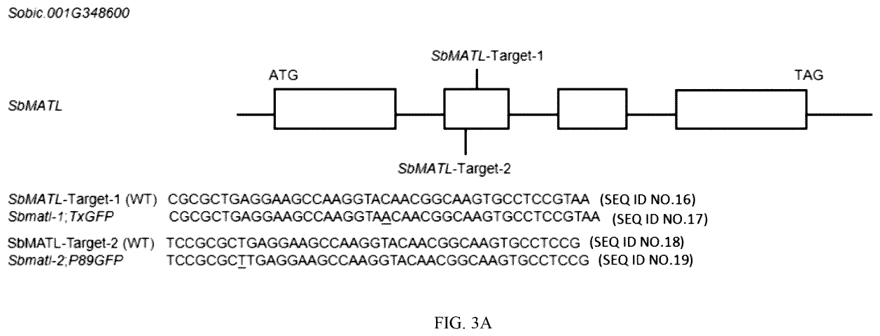

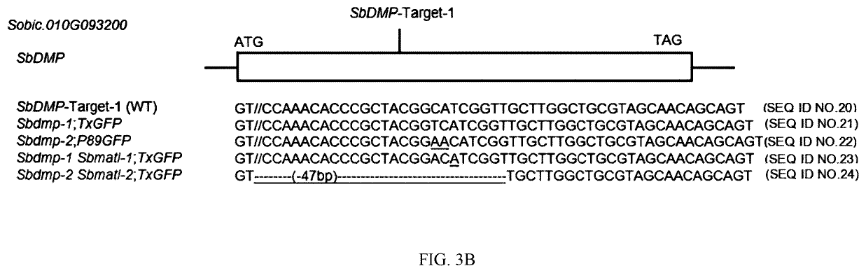

(1) Selection of sgRNA target site sequences: two target sites, SbMTL-sgRNA1 and SbMTL-sgRNA2, are selected on the Sorghum SbMTL genome; the sequence of SbMTL-sgRNA1 is CTGAGGAAGCCAAGGTACAA (SEQ ID NO.13), located at positions 450-469 on SEQ ID NO.1; the sequence of SbMTL-sgRNA2 is CGCTGAGGAAGCCAAGGTAC (SEQ ID NO.14), located at positions 448-467 on SEQ ID NO.1. One target site, SbDMP-sgRNA1, is selected on the Sorghum SbDMP genome, with its sequence being CCAAACACCCGCTACGGCAT (SEQ ID NO.15), located at positions 601-620 on SEQ ID NO.3. Specific gene structures and target site information are shown in FIG. 3A and FIG. 3B.

FIG. 3A shows information on two target sites on the SbMTL gene, respectively obtaining editing types of two homozygous frameshift mutant lines. FIG. 3B shows information on a single target site on the SbDMP gene, obtaining editing types of single mutant lines and double mutants. On the FIG. 3A and FIG. 3B, underlined font represents the edited base type.

(2) Construction of CRISPR/Cas9 vectors: the vectors CRISPR/SbMTL-Cas9-1, CRISPR/SbMTL-Cas9-2, and CRISPR/SbDMP-Cas9-1 are constructed by respectively inserting the target site DNA (20 nucleotides) sequences of SbMTL-sgRNA1, SbMTL-sgRNA2, and SbDMP-sgRNA1 into the PCas9 vector (Miao, Guo et al. 2013) (refer to Qu Lijia's Cell Research paper, which is publicly available and is an open-access vector).

(3) Acquisition of transgenic Sorghum: the vectors CRISPR/SbMTL-Cas9-1, CRISPR/SbMTL-Cas9-2, and CRISPR/SbDMP-Cas9-1 obtained in the step (2) are transformed into Agrobacterium competent cells EHA105 (purchased from Shanghai Weidi Biotechnology Co., Ltd.); and Agrobacterium containing positive CRISPR/SbMTL-Cas9-1, CRISPR/SbMTL-Cas9-2, and CRISPR/SbDMP-Cas9-1 is transformed into Sorghum P89GFP and TxGFP recipients, and after immature embryo transformation, screening, differentiation, and rooting, T₀ generation transgenic positive Sorghum plants are obtained.

(4) Identification of Sorghum SbMTL and SbDMP single-gene mutant and double-gene mutant lines: DNA is extracted from the leaves of T₀ generation positive plants from step (3), and the following primers (amplification primers are SEQ ID NO.7 to SEQ ID NO.10; and sequencing primers are SEQ ID NO.11 and SEQ ID NO.12) are used for target site PCR amplification, and the amplification products are sequenced.

The sequencing results are compared with the wild-type SbMTL and SbDMP genes, respectively, and the presence and type of mutations in SbMTL and SbDMP are identified in different lines of T₀ generation transgenic Sorghum. Mutant plants are grown to produce T₁ generation plants, and stably inherited T₂ seeds are obtained. In T₁ plants, plants from which the CRISPR/SbMTL-Cas9-1 or CRISPR/SbMTL-Cas9-2 vectors are excised are identified and preserved for use in creating double mutant lines.

The mutant lines subsequently used for evaluating haploid induction capability in the present disclosure are shown in FIG. 3A and FIG. 3B. These include SbMTL single mutant lines sbmtl-1; TxGFP and sbmtl-2; P89GFP, SbDMP single mutant lines sbdmp-1; TxGFP and sbdmp-2; P89GFP, and double mutant lines sbdmp-1 sbmtl-1; TxGFP and sbdmp-2 sbmtl-2; P89GFP in which SbDMP is further edited in the sbmtl-1 and sbmtl-2 mutant plants.

SbMTL mutation sequence detection primers: SbMTL-F: CTCATCACCGCCATGCTCACCG (SEQ ID NO.7) and SbMTL-R: CGTCGCTGTGGAGCGACTG (SEQ ID NO.8).

SbDMP mutation sequence detection primers: SbDMP-F: TCTTCCACTTCACCGACAGCTT (SEQ ID NO.9) and SbDMP-R: CCCTGGCCCCTTCATGATACAT (SEQ ID NO.10).

Sequencing primers: SbMTL-sequence-F: CAAGGACAGGCGGCCTCTCTACG (SEQ ID NO.11) and SbDMP-sequence-F: GGCAAGGTCTACTACGGCTT (SEQ ID NO.12).

### Embodiment 4

### Method for Producing High-Frequency Haploids in Sorghum

Six mutant lines in two backgrounds obtained previously and two base backgrounds are included: two SbMTL single mutants (sbmtl-1; TxGFP and sbmtl-2; P89GFP), two SbDMP single mutants (sbdmp-1; TxGFP and sbdmp-2; P89GFP), and two SbMTL SbDMP double mutants (sbdmp-1 sbmtl-1; TxGFP and sbdmp-2 sbmtl-2; P89GFP). The two base backgrounds P89GFP, TxGFP (created previously) and the Sorghum male sterile line L407A (gifted by Researcher Li Guiying from the Institute of Crop Science, Chinese Academy of Agricultural Sciences, and available to the public) make a total of nine materials used as experimental materials for field evaluation of haploid induction capability, and the specific steps are as follows.

(1) The nine materials are sown at the same time, and the eight materials other than the sterile line L407A are sown in two additional phases to ensure flowering synchronization with the sterile line L407A. After all materials heading, bagging is strictly performed. The eight lines are used as pollen donors and crossed with the sterile line L407A to obtain hybrid seeds. After germination of the hybrid seeds, identification is carried out based on the presence or absence of GFP in the Sorghum seed embryos and leaf ploidy determination by flow cytometry. Specific results are shown in Table 1.

TABLE 1
Evaluation of Sorghum haploid induction frequency

| | | | | Total | | | Average |
|---|---|---|---|---|---|---|---|
| Exper- | Female | | Total | germinated | Total | Induction | induction |
| iment | parent in | | hybrid | hybrid | haploid | rate | rate |
| number | cross | Male parent in cross | seeds | plants | count | (%) | (%) |
| 1 | L407A | sbdmp-1; TxGFP | 156 | 154 | 0 | 0.00 | 0.00 |
| 2 | L407A | sbdmp-1; TxGFP | 344 | 344 | 0 | 0.00 | |
| 3 | L407A | sbdmp-1; TxGFP | 500 | 498 | 0 | 0.00 | |
| 4 | L407A | sbmtl-1: TxGFP | 1036 | 969 | 13 | 1.34 | 1.95 |
| 5 | L407A | sbmtl-1; TxGFP | 1132 | 1082 | 18 | 1.66 | |
| 6 | L4074 | sbmtl-1: TxGFP | 380 | 352 | 10 | 2.84 | |
| 7 | L407A | sbdmp-1 sbmtl-1; TxGFP | 231 | 198 | 13 | 6.57 | 7.38 |
| 8 | L407A | sbdmp-1 sbmtl-1, TxGFP | 354 | 291 | 31 | 10.65 | |
| 9 | L407A | sbdmp-1 sbmtl-1; TxGFP | 283 | 264 | 13 | 4.92 | |
| 10 | L407A | sbdmp-2; P89GFP | 478 | 421 | 1 | 0.24 | 0.06 |
| 11 | L407A | sbdmp-2; P89GFP | 359 | 311 | 0 | 0.00 | |
| 12 | L407A | sbamp-2: P89GFP | 657 | 580 | 0 | 0.00 | |
| 13 | L407A | sbdmp-2; P89GFP | 463 | 417 | 0 | 0.00 | |
| 14 | L407A | sbmtl-2; P89GFP | 891 | 891 | 40 | 4.49 | 6.98 |
| 15 | L407A | sbmtl-2; P89GFP | 307 | 307 | 21 | 6.84 | |
| 16 | L407A | sbmtl-2; P89GFP | 1231 | 996 | 50 | 5.02 | |
| 17 | L407A | sbmtl-2; P89GFP | 337 | 236 | 25 | 10.59 | |
| 18 | L4074 | sbmtl-2; P89GFP | 276 | 220 | 16 | 7.27 | |
| 19 | L407A | shmtl-2; P89GFP | 349 | 256 | 12 | 4.69 | |
| 20 | L407.A | sbmtl-2; P89GFP | 207 | 164 | 13 | 7.93 | |
| 21 | L407A | sbmtl-2; P89GFP | 369 | 278 | 25 | 8.99 | |
| 22 | L407A | sbdmp-2 sbmtl-2: P89GFP | 635 | 458 | 66 | 14.41 | 18.94 |
| 23 | L407A | sbdmp-2 sbmtl-2; P89GFP | 334 | 274 | 47 | 17.15 | |
| 24 | L407A | sbdmp-2 sbmtl-2; P89GFP | 159 | 122 | 15 | 12.30 | |
| 25 | L407A | sbdmp-2 sbmtl-2; P89GFP | 123 | 66 | 19 | 28.79 | |
| 26 | L407A | sbdmp-2 sbmtl-2: P8PGFP | 96 | 68 | 15 | 22.06 | |

As shown in Table 1, after mutating the SbMTL gene alone in the two backgrounds, the haploid induction rates are 1.95% (TxGFP) and 6.98% (P89GFP), respectively. Mutating the SbDMP gene alone shows no induction frequency in the TxGFP background, while the induction frequency in the P89GFP background is extremely low at only 0.06%. When sbdmp mutation is superimposed on sbmtl mutation, the induction rates in the two backgrounds are 7.38% (TxGFP) and 18.94% (P89GFP), respectively. Particularly important is that in the P89GFP background, the induction rate increases from 6.98% (202 haploids identified from 3,967 hybrids) for sbmtl single mutant to 18.94% (162 haploids identified from 1,347 hybrids) for sbmtl sbdmp double mutant. Meanwhile, no haploids are observed when wild-type TxGFP and P89GFP are used as controls (observed from 1,491 hybrids).

(2) Determination of haploid phenotype is carried out by flow cytometry.

All haploids shown in Table 1 above undergo leaf ploidy identification by flow cytometry, and the specific method is as follows.

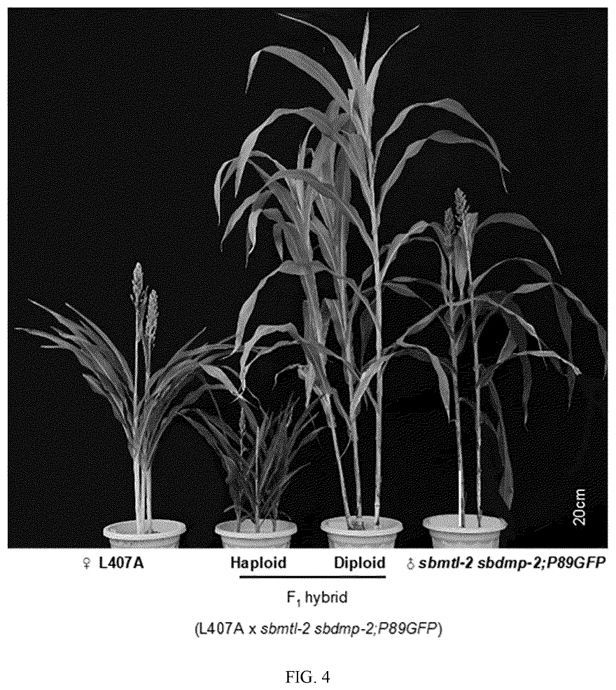

The above hybrid seeds are planted in the field, and individual plant phenotypes are observed, as shown in FIG. 4 (from left to right: female parent sterile line L407A, haploids (Haploid) and diploids (Diploid) appearing in the hybrid offspring of L407A and the induction line, and the induction line sbmtl-2 sbdmp-2; P89GFP): diploid plants exhibit heterosis, with taller plants, broader leaves, and normal fertility, while hybrid haploid plants are dwarf, have narrower leaves, compact plant architecture, and are sterile. Based on these characteristics, potential haploids are preliminarily judged.

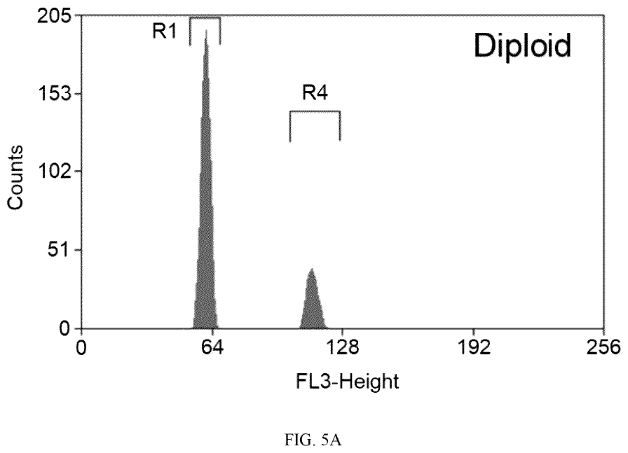

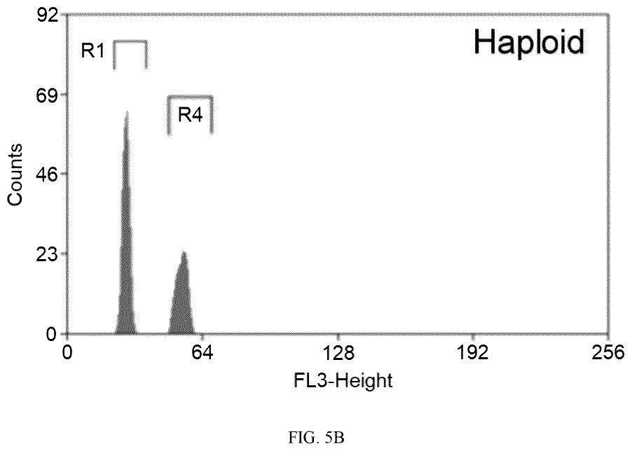

Nuclei from young leaves of these potential haploid plants are collected, with leaves from diploid Sorghum wild-type plants P89GFP or TxGFP used as controls. The signals are then detected using a flow cytometer. The diploid nuclear signal is detected first, and the diploid nuclear signal peak position is set at 64 (since the genetic material in diploid cells is twice that in haploid cells, the haploid nuclear signal peak is theoretically expected to appear near 32). If the signal peak of the tested plant appears near 64, it is considered to have the same signal intensity enrichment position as the diploid nuclear signal, and the tested plant is classified as a diploid. If the nuclear signal peak of the tested plant appears near 32, the tested plant is considered a haploid plant. The detection results are shown in FIG. 5A and FIG. 5B. The error in flow cytometer measurements is small, so after measurement, the tested plants may be accurately classified into haploid and diploid groups.

Based on Embodiments 1 to 4, it is concluded that mutating Sorghum SbMTL and superimposing SbDMP mutation may produce high-frequency haploids, with the average induction efficiency of the induction line in the P89GFP background reaching 18.94%, which provides the possibility for industrial production of Sorghum haploids.

### Embodiment 5

### Sorghum Haploid Screening and Identification Methods

(1) Identification of Sorghum haploids using the GFP marker:

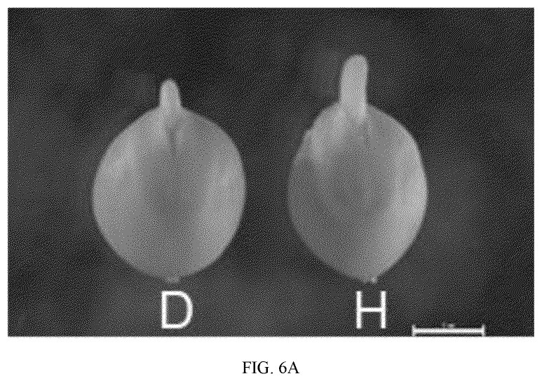

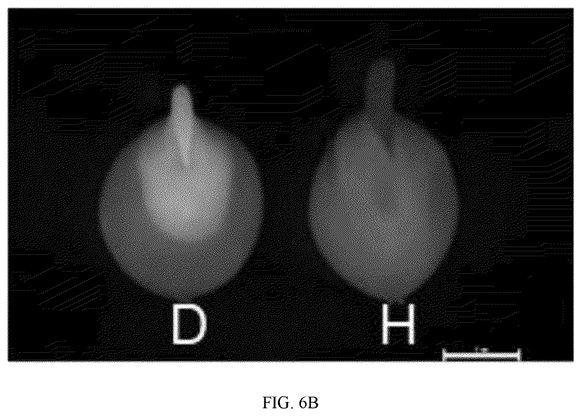

In the induction lines in the TxGFP and P89GFP backgrounds, the plants contain a single-copy GFP marker. When the induction line is crossed with the maternal parent to be induced, true hybrid embryos carry the GFP marker from the paternal parent, and under ultraviolet light, hybrid embryos display green fluorescence, while haploids lack GFP fluorescence. Therefore, haploids and diploids may be distinguished by fluorescence contrast, with specific fluorescence and white light comparison images shown in FIG. 6A and FIG. 6B. FIG. 6A shows a comparison under white light between Sorghum diploids (D) and haploids (H) after pollination with the induction line, and FIG. 6B shows a comparison of diploids (D) and haploids (H) under ultraviolet light.
(2) Introduction of the Ruby reporter gene into haploid induction lines for haploid identification:

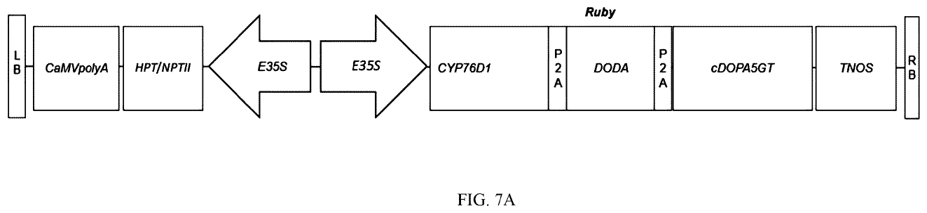

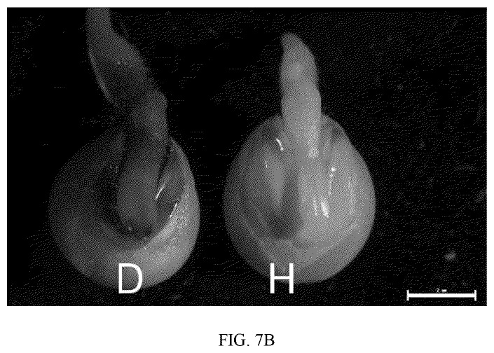

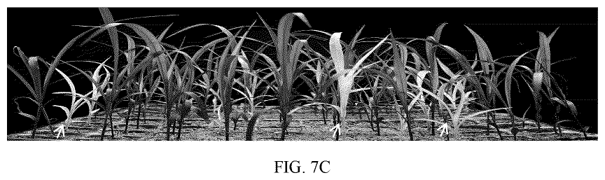

An expression cassette containing Ruby (FIG. 7A is a schematic diagram of the expression vector structure of Ruby transformed into the induction line) is introduced into the induction line via Agrobacterium-mediated genetic transformation, and T₂ generation single-copy homozygous lines Ruby-inducer are obtained. The Ruby-inducer line is crossed with a sterile line, and the presence of haploids may be visually determined by observing the presence or absence of Ruby in the resulting hybrids. As shown in FIG. 7B, after germination of seeds from crossing between the induction line containing the Ruby marker and the female parent, diploids (D) and haploids (H) may be distinguished by the presence or absence of red markers in the plumule, where the markers in the plumule of the diploid (D) appear as a visually darker region. FIG. 7C shows that Sorghum seeds after crossing with the induction line are directly sown in soil, and the growing plants may be identified for haploids using the red Ruby marker, with green (a lighter color) indicating haploids (shown by white arrows) and all red plants (a darker color) being diploids.

### Embodiment 6

### Natural Doubling of Sorghum Haploids to Obtain Doubled Haploids (DH₀)

The final step in doubled haploid breeding is the doubling of the haploid plant genome to generate diploids for subsequent trait evaluation. Although spontaneous chromosome doubling occasionally occurs in haploid maize plants, chemicals such as colchicine are typically required for large-scale production of doubled haploid plants.

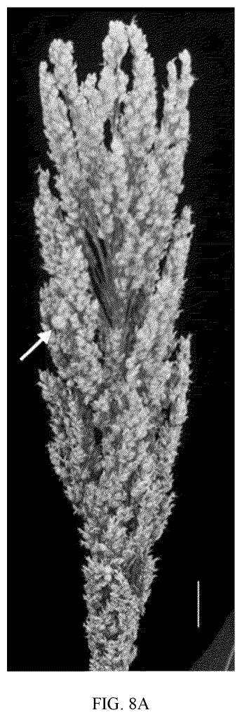

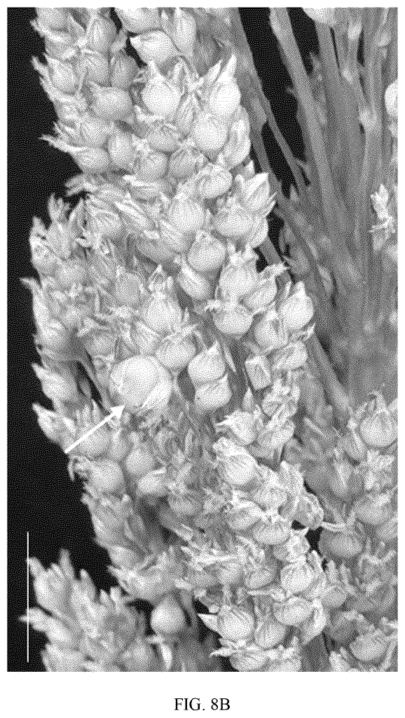

To investigate whether haploid Sorghum plants may obtain doubled haploids through natural doubling, experiments are conducted by artificial emasculation to prevent the induced parent from self-pollination, and then the induction line is used to pollinate the emasculated parent, resulting in haploids consistent with the maternal parent. The results show that among 60 haploid plants, 59 are able to spontaneously produce seeds of DH₀ in varying quantities. As shown in FIG. 8A and FIG. 8B, seeds of DH₀ produced by natural doubling of Sorghum haploids are presented, where FIG. 8A shows the panicle of a Sorghum haploid, with individual florets capable of natural doubling to produce seeds of DH₀; and FIG. 8B is a partial enlargement of FIG. 8A, with seeds of DH₀ indicated by white arrows.

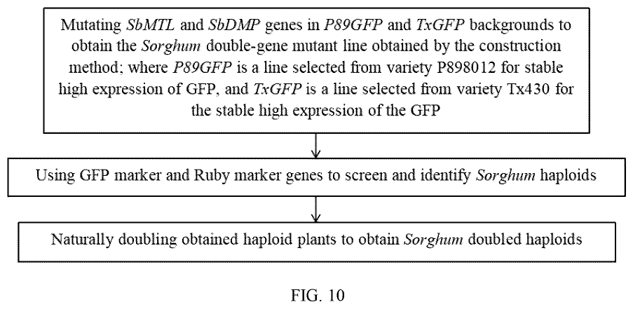

As shown in FIG. 10, these results indicate that Sorghum haploids may obtain doubled haploids through natural doubling.

The above description is only the optional embodiment of the present disclosure and is not intended to limit the present disclosure. Although the present disclosure has been described in detail with reference to the foregoing embodiments, those skilled in the art may still modify the technical schemes described in the foregoing embodiments or perform equivalent replacements of some of the technical features. Any modifications, equivalent replacements, improvements, etc., made within the spirit and principles of the present disclosure shall be included within the protection scope of the present disclosure.

What is claimed is:

1. A construction method for a Sorghum double-gene mutant line, wherein the construction method utilizes clustered regularly interspaced short palindromic repeats/CRISPR-associated protein 9 (CRISPR/Cas9) technology to knock out Sorghum SbMTL and SbDMP genes respectively in P89GFP and TxGFP backgrounds, with a genomic sequence of SbMTL being as shown in SEQ ID NO.1, and a genomic sequence of SbDMP being as shown in SEQ ID NO.3; and obtained SbMTL and SbDMP double-gene mutant lines comprise two knockout lines: sbdmp-1 sbmtl-1; TxGFP and sbdmp-2 sbmtl-2; P89GFP; and
wherein P89GFP is a line selected from variety P898012 for stable high expression of green fluorescent protein (GFP), and TxGFP is a line selected from variety Tx430 for the stable high expression of the GFP.

2. The construction method for the Sorghum double-gene mutant line according to claim 1, comprising following steps:
selecting a target site SbMTL-sgRNA1 with a sequence as shown in SEQ ID NO.13 and a target site SbMTL-sgRNA2 with a sequence as shown in SEQ ID NO.14 on a Sorghum SbMTL genome; and selecting a target site SbDMP-sgRNA1 with a sequence as shown in SEQ ID NO.15 on a Sorghum SbDMP genome;
constructing CRISPR/SbMTL-Cas9-1, CRISPR/SbMTL-Cas9-2, and CRISPR/SbDMP-Cas9-1 vectors comprising target site DNA sequences; and
using Agrobacterium to transform the vectors into Sorghum P89GFP and TxGFP receptors to obtain transgenic Sorghum with knocked-out SbMTL and SbDMP genes, thereby obtaining the Sorghum double-gene mutant line.

3. The construction method for the Sorghum double-gene mutant line according to claim 1, wherein in the P89GFP background, the SbDMP gene on SbMTL single mutant plants is knocked out to obtain sbmtl sbdmp double-gene mutant haploid induction lines.

4. A Sorghum doubled haploid breeding method, comprising following steps:
(1) mutating SbMTL and SbDMP genes in P89GFP and TxGFP backgrounds to obtain the Sorghum double-gene mutant line obtained by the construction method according to claim 1; wherein P89GFP is a line selected from variety P898012 for stable high expression of GFP, and TxGFP is a line selected from variety Tx430 for the stable high expression of the GFP;
(2) using GFP marker and Ruby marker genes to screen and identify Sorghum haploids; and
(3) naturally doubling obtained haploid plants to obtain Sorghum doubled haploids.


SEQUENCE LISTING (15 sequences)

SEQ ID NO: 1 - DNA, 1588 bp, synthetic construct
  source 1..1588: mol_type = other DNA
atgcaacgcctgcagcacgaaggcgatggccgggagcgtggtcggcgagcccgtcgtgctggggcagagggtgacggtgctgacggtggacggcggcggcatccgtggtctcatccccggaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagtgagtccgatcatcccggccggccgccattgttggcctcgcattattgatcgatcatgcatgcatgcatgcatgtgatgtgtaggagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgtacgtacgccgccggccgccgtcgtcatgaataatcaatcagctgtacattattgcacatggctgcatgcctactgaacatgtttaatttgcaggccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccggtaattaattaactgaaaacgaacgaacgcaattccatatccattgtccagaagatcgatgctaattgttgcttaggcacgtcgttgccgccggccgattgcattgcacttgcacatgcgtagacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

SEQ ID NO: 2 - DNA, 1314 bp, synthetic construct
  source 1..1314: mol_type = other DNA
atggcgacctactactcttcgcggcgtccatgcaacgcctgcagcacgaaggcgatggccgggagcgtggtcggcgagcccgtcgtgctggggcagagggtgacggtgctgacggtggacggcggcggcatccgtggtctcatccccggaaccatccttgccttcctcgaggcccggctgcaggagctggacgggccggaggttaggctcgcggactacttcgactacatcgccgggacgagcaccggcgggctcatcaccgccatgctcaccgcgcccggcaaggacaggcggcctctctacgctgccaaggacatcaaccaattctacatggagaattgccctcgcatcttccctcaaaagagcagcaggcttgcggccgccatgtccgcgctgaggaagccaaggtacaacggcaagtgcctccgtaacctgatcatgagcatgctcggcgagacgagggtgagcgacacgctcaccaacgtcatcatccctaccttcgacgtcaggctgctgcagcccatcatcttctccacctacgacgccaagagcatgcctctgaagaacgcgctgctctccgacgtgtgcatcggcacgtccgccgcgccgacctacctcccggcgcactacttccagaccaaggacgccggcagtggcaaggaacgcgagtacaacctcatcgacggcggtgtcgccgccaacaatccgacgatggttgcgatgacgcagatcaccaagaagatgcttgccagcaaggagaaggccgaggagctgtacccagtgaagccgtggaactgccgcaagttcctggtgctgtccatcgggacggggtcgacgtcggagcagggcctgtacacggcgcggcagtgctcgcggtggggcatctgccggtggatccggaacaacggcatggcccccatcatcgacatcttcatggcggcgagctcggacctggtggacatccacgtcgccgcgatgttccagtcgctccacagcgacggcgactacctgcgcatccaggacaactcgctgcacggcgccgcggccaccgtggacgcggcgacgccggagaacatgcggacgctcgtcgggatcggggagcggatgctggcgcagcgggtgtccagggtcaacgtggagacagggaggtacgaaccggtgcctggggaaggaagcaacgctgatgcgctcgctgggatcgcaaggcagctctcggaggagaggaggacaaggctcgcgcgccgcacctccgccatcgtcagctccggtggtgcctctagacgtacgtgtgcctcaaaggtctccaatgtctaa

SEQ ID NO: 3 - DNA, 639 bp, synthetic construct
  source 1..639: mol_type = other DNA
atggaacgcagcaacgtcgccgcggccggtgcggtgtccgtcgaggtgctccgcgcccgcggcggcggcggctcgccgccggcgggcgcggggaggaagcgccgcttggtggcgaggggcgtccagagcacgctctccaagacgtccatgctggccaacttcctccccacgggcacgctgctgaccttcgagatgctcctcccggccgcctcaggcgacggcacctgctcggcggtcagcgtcgcgatgctcagggcgctcctcgcgctctgcgccgcgtcctgcttcctcttccacttcaccgacagcttccgcgcgccggacggcaaggtctactacggcttcgtcacgccgggcgggctgtcgctgttcaggaccgggctcgacggcgtcgaggtgcccagggaggagaggtaccggctcgccttcgtcgacgtcgtgcacgccgtcatgtcggtgctggtcttcgcggccgtcgcgctcgcggactaccgcgtctccgggtgcctcgtcgccggccaccgcaaggagatgggcgaggtgatggagagcttcccgctcatggtgggcgccgtgtgcagcggcctcttcctgctgttcccaaacacccgctacggcatcggttgcttggctgcgtag

SEQ ID NO: 4 - DNA, 639 bp, synthetic construct; identical to SEQ ID NO: 3
  source 1..639: mol_type = other DNA

SEQ ID NO: 5 - protein, 437 aa, synthetic construct
MATYYSSRRPCNACSTKAMAGSVVGEPVVLGQRVTVLTVDGGGIRGLIPGTILAFLEARLQELDGPEVRLADYFDYIAGTSTGGLITAMLTAPGKDRRPLYAAKDINQFYMENCPRIFPQKSSRLAAAMSALRKPRYNGKCLRNLIMSMLGETRVSDTLTNVIIPTFDVRLLQPIIFSTYDAKSMPLKNALLSDVCIGTSAAPTYLPAHYFQTKDAGSGKEREYNLIDGGVAANNPTMVAMTQITKKMLASKEKAEELYPVKPWNCRKFLVLSIGTGSTSEQGLYTARQCSRWGICRWIRNNGMAPIIDIFMAASSDLVDIHVAAMFQSLHSDGDYLRIQDNSLHGAAATVDAATPENMRTLVGIGERMLAQRVSRVNVETGRYEPVPGEGSNADALAGIARQLSEERRTRLARRTSAIVSSGGASRRTCASKVSNV

SEQ ID NO: 6 - protein, 212 aa, synthetic construct
MERSNVAAAGAVSVEVLRARGGGGSPPAGAGRKRRLVARGVQSTLSKTSMLANFLPTGTLLTFEMLLPAASGDGTCSAVSVAMLRALLALCAASCFLFHFTDSFRAPDGKVYYGFVTPGGLSLFRTGLDGVEVPREERYRLAFVDVVHAVMSVLVFAAVALADYRVSGCLVAGHRKEMGEVMESFPLMVGAVCSGLFLLFPNTRYGIGCLAA

SEQ ID NO: 7 - DNA, 22 bp, synthetic construct
  source 1..22: mol_type = other DNA
ctcatcaccgccatgctcaccg

SEQ ID NO: 8 - DNA, 19 bp, synthetic construct
  source 1..19: mol_type = other DNA
cgtcgctgtggagcgactg

SEQ ID NO: 9 - DNA, 22 bp, synthetic construct
  source 1..22: mol_type = other DNA
tcttccacttcaccgacagctt

SEQ ID NO: 10 - DNA, 22 bp, synthetic construct
  source 1..22: mol_type = other DNA
ccctggccccttcatgatacat

SEQ ID NO: 11 - DNA, 23 bp, synthetic construct
  source 1..23: mol_type = other DNA
caaggacaggcggcctctctacg

SEQ ID NO: 12 - DNA, 20 bp, synthetic construct
  source 1..20: mol_type = other DNA
ggcaaggtctactacggctt

SEQ ID NO: 13 - DNA, 20 bp, synthetic construct
  source 1..20: mol_type = other DNA
ctgaggaagccaaggtacaa

SEQ ID NO: 14 - DNA, 20 bp, synthetic construct
  source 1..20: mol_type = other DNA
cgctgaggaagccaaggtac

SEQ ID NO: 15 - DNA, 20 bp, synthetic construct
  source 1..20: mol_type = other DNA
ccaaacacccgctacggcat